(19) United States
(12) Patent Application Publication
(10) Pub. No.: US 2016/0088788 A1
(43) Pub. Date: Mar. 31, 2016

(54) Biogas Production from a Flexible Digester

(71) Applicant: Dominic F.P. Wanjihia, Nairobi (KE)

(72) Inventor: Dominic F.P. Wanjihia, Nairobi (KE)

(21) Appl. No.: 14/710,623

(22) Filed: May 13, 2015

Related U.S. Application Data
(63) Continuation of application No. PCT/KE2013/000031, filed on Apr. 8, 2013.

(30) Foreign Application Priority Data
Nov. 14, 2012 (KE) KE/P/2012/001702

(51) Int. Cl. A01C 3/02; C12M 1/00

(52) U.S. Cl. CPC A01C 3/023; C12M 23/26; C12M 29/20; C12M 29/00

(57) ABSTRACT

In one aspect, the invention provides an apparatus for anaerobic digestion of digester feed, the apparatus having: (a) a flexible tubular bladder; (b) a digester feed inlet port and tube; (c) a slurry outlet port and tube; and (d) a gas outlet port and tube. The invention also provides a method for using such an apparatus and similar apparatus described within.

### CROSS-REFERENCE TO RELATED APPLICATIONS

This application claims priority to PCT application serial number PCT/KE2013/000031, filed Apr. 8, 2013, which claims priority to Kenya patent application KE/P/2012/001702, filed Nov. 14, 2012. The contents of the aforementioned applications are incorporated herein in their entireties.

### BACKGROUND

Two types of biogas digesters have received widespread exposure, the Floating Dome type, and the Fixed Dome type. Studies have revealed several reasons for these systems not been widely adopted. For example, both systems are very expensive to install. The systems are typically fixed capacity and non-expandable. The systems may require absolute precision technical expertise in construction. Frequently, metal parts of the dome are prone to rusting and require regular repair or replacement. In most all cases, due to the construction requirements, the installer must own the land. For at least these reasons, biogas digesters remain minor in terms of world-wide energy production.

Relevant Art: US 2007/0256971, U.S. Pat. No. 6,929,744, WO 2009/073902, and EP 1100867.

### SUMMARY

In one aspect, there is provided an apparatus for anaerobic digestion of digester feed, the apparatus comprising: (a) a flexible tubular bladder comprising: (i) a top side comprising a gas outlet port; (ii) a bottom side comprising a digester feed inlet port and a slurry outlet port; (iii) a digester feed end sealed with a digester feed end seal; and (iv) a slurry end sealed with a slurry end seal, provided that at least one of the digester feed end seal and the slurry end seal is a removable seal; (b) a digester feed inlet tube connected to the digester feed inlet port; (c) a slurry outlet tube connected to the slurry outlet port; and (d) a gas outlet tube connected to the gas outlet port.

In embodiments:

the connection of the inlet tube to the inlet port is not gastight, and wherein the connection of the outlet tube to the outlet port is not gastight;

the digester feed end seal is not gastight and is not liquid tight, and wherein the slurry end seal is not gastight and is not liquid tight;

the digester feed end seal comprises a single or double zip, and wherein the slurry end seal comprises a single or double zip;

the digester feed end seal is a removable seal;

the slurry end seal is a removable seal;

the digester feed end seal and the slurry end seal are both removable seals;

the digester feed inlet tube has a diameter, the slurry outlet tube has a diameter, and the digester feed inlet tube diameter is equal to or smaller than the slurry outlet tube diameter;

the digester feed inlet tube comprises a horizontal section, a feed section that connects to the horizontal section at an oblique angle, and a rise section that connects to the horizontal section at substantially a right angle, wherein the rise section forms the connection of the digester feed inlet tube to the digester feed inlet port;

the digester feed inlet tube comprises a descending section intersecting an ascending section to form an angle in the range 50-130 degrees, wherein the ascending section forms the connection of the digester feed inlet tube to the digester feed inlet port;

the slurry outlet tube comprises a horizontal section, a takeoff section that connects to the horizontal section at an oblique angle, and a drop section that connects to the horizontal section at substantially a right angle, wherein the drop section forms the connection of the slurry outlet tube to the slurry outlet port;

the flexible tubular bladder is made of a flexible non-elastic film;

the apparatus comprises an auxiliary biogas collection container connected to the gas outlet tube;

the apparatus further comprises a greenhouse configured to enclose the tubular container;

the apparatus further comprises one or more heating pipes within or under the flexible tubular bladder;

the connection of the inlet tube to the inlet port is not gastight, the connection of the outlet tube to the outlet port is not gastight, the digester feed end seal is not gastight and is not liquid tight, and the slurry end seal is not gastight and is not liquid tight;

the digester feed inlet tube diameter is smaller than the slurry outlet tube diameter, the digester feed inlet tube comprises a horizontal section, a feed section that connects to the horizontal section at an oblique angle, and a rise section that connects to the horizontal section at a right angle, and the slurry outlet tube comprises a horizontal section, a takeoff section that connects to the horizontal section at an oblique angle, and a drop section that connects to the horizontal section at a right angle;

the flexible tubular bladder is made of a flexible non-elastic film, and wherein the digester feed inlet tube diameter is the same or smaller than the slurry outlet tube diameter;

the flexible tubular bladder is made of a flexible non-elastic film, and wherein the apparatus further comprises one or more heating pipes within or under the flexible tubular bladder; and

the flexible tubular bladder is made of a flexible film, and wherein the digester feed inlet tube comprises a horizontal section, a feed section that connects to the horizontal section at an oblique angle, and a rise section that connects to the horizontal section at substantially a right angle, and the slurry outlet tube comprises a horizontal section, a takeoff section that connects to the horizontal section at an oblique angle, and a drop section that connects to the horizontal section at substantially a right angle.

In another aspect, there is provided a method for anaerobically digesting digester feed with biogas production, comprising conveying the digester feed into an apparatus as described above.

In embodiments:

The conveying involves adding the digester feed into the digester feed inlet tube, wherein the digester feed may comprise a mixture of digester feed and water sufficient to: (a) hydrate the contents of the apparatus; and (b) facilitate flow of the digester feed through the apparatus;

the method involves removing biogas produced within the apparatus through the gas outlet tube; and

the method involves installing the apparatus prior to conveying, wherein the installation comprises: (a) leveling the ground or otherwise providing a level surface large enough to accommodate the apparatus; (b) providing recessed areas (i.e., recessed below the level of the ground or the level surface) for the digester feed inlet tube and for the slurry outlet tube; and (c) priming the apparatus by adding a substrate containing bacteria (e.g., cow dung, etc.).

In another aspect, there is provided a method for anaerobically digesting digester feed with biogas production, comprising the steps of: (i) conveying the digester feed through a digester feed inlet tube and into a flexible tubular bladder via a digester feed inlet port, wherein the digester feed inlet port is located on a bottom side of the flexible tubular bladder at a digester feed end; (ii) causing the digester feed to travel from the digester feed end of the flexible tubular bladder to a slurry end of the flexible tubular bladder over a period of time, wherein digestion of digester feed and production of biogas occurs during the period of time; and (iii) removing slurry from the flexible tubular bladder through a slurry outlet tube connected to a slurry outlet port, wherein the slurry outlet port is located on the bottom side of the flexible tubular bladder at the slurry end.

In embodiments:

the flexible tubular bladder is made of a flexible film;

the causing of (ii) comprises conveying additional digester feed into the flexible tubular bladder;

the method further comprises maintaining the digester feed and slurry (i.e., the substrate) in the tubular bladder at an average temperature of 25 degrees Celsius or greater during the period of time;

the maintaining is carried out passively;

the method further comprises removing biogas from the flexible tubular bladder via a gas outlet tube connected to a gas outlet port, wherein the gas outlet port is located on a top side of the flexible tubular bladder;

the flexible tubular bladder is made of a flexible film, and wherein the method further comprises maintaining the digester feed and slurry (i.e., the substrate) in the tubular bladder at an average temperature of 25° C. or greater during the period of time; and

the flexible tubular bladder is made of a flexible film, and wherein the method further comprises removing biogas from the flexible tubular bladder via a gas outlet tube connected to a gas outlet port, wherein the gas outlet port is located on a top side of the flexible tubular bladder.

Any combination of the embodiments described herein is envisioned within the scope of the invention, and this statement provides antecedent basis for combining elements as if such combinations were laboriously and explicitly set forth.

These and other aspects of the invention will be apparent to those of skill in the art from the description provided herein. The technical field(s) of the invention is/are the mechanical and environmental arts.

### BRIEF DESCRIPTION OF THE VIEW OF THE DRAWING

FIG. 1 is a drawing showing a flexible tubular bladder with a double zip according to one embodiment of the invention.

### DETAILED DESCRIPTION OF THE VARIOUS EMBODIMENTS

The inventive apparatus may be called a “digester” or simply an “apparatus.” The term “substrate” refers to the contents of the inventive apparatus when in use, and includes both digester feed and slurry.

In one aspect, there is provided an apparatus for anaerobic digestion of digester feed, the apparatus comprising: (a) a flexible tubular bladder comprising: (i) a top side comprising a gas outlet port; (ii) a bottom side comprising a digester feed inlet port and a slurry outlet port; (iii) a digester feed end sealed with a removable digester feed end seal; and (iv) a slurry end sealed with a removable slurry end seal; (b) a digester feed inlet tube connected to the digester feed inlet port; (c) a slurry outlet tube connected to the slurry outlet port; and (d) a gas outlet tube connected to the gas outlet port.

During use of the apparatus, all seals and ports (i.e., port connections) that are exposed to the generated biogas must be gastight, and failure of the gastight seals and/or ports causes loss of generated biogas. Prior to use, in embodiments, one or more of the various seals and ports of the apparatus is/are not gastight. This is possible because such seals and ports become gastight over the course of normal use. Solid materials from the digester feed and slurry accumulate and block any escape paths for gas. Such sealing of the seals and ports occurs to any seal and port that is located below the liquid level within the apparatus. Thus, in embodiments, the inlet port, the outlet port, the digester feed end, and the slurry end are all manufactured without gastight seals and become gastight with use of the apparatus. In other embodiments, any one or more of such seals and ports are manufactured with gastight seals (e.g., using rubber O-rings, etc.).

The gas outlet port is located on the top side of the apparatus, and therefore is not typically below the level of liquid within the apparatus. Accordingly, the gas outlet port and gas outlet tube are manufactured to form a gastight seal, and such gastight seal is present even before actual use of the apparatus.

During use of the apparatus, all seals and ports (i.e., port connections) that are exposed to liquid(s) must be (or must become) liquid tight, as failure of the liquid tight seals and/or ports causes leaking of digester feed and/or slurry. Prior to use, in embodiments, one or more of the various seals and ports of the apparatus is/are not liquid tight. This is possible because such seals and ports become liquid tight over the course of normal use. Solid materials from the digester feed and slurry accumulate and block any escape paths for liquid. Such sealing of the seals and ports occurs to any seal and port that is located below the liquid level within the apparatus. Thus, in embodiments, the inlet port, the outlet port, the digester feed end, and the slurry end are all manufactured without liquid tight seals and become liquid tight with use of the apparatus. In other embodiments, any one or more of such seals and ports are manufactured with liquid tight seals.

In embodiments, the connection of the inlet tube to the inlet port is not gastight. In embodiments, the connection of the outlet tube to the outlet port is not gastight. In embodiments, both such connections are not gastight.

In embodiments, the removable digester feed end seal is not gastight and is not liquid tight. In embodiments, the removable slurry end seal is not gastight and is not liquid tight. In embodiments, both such seals are not gastight and are not liquid tight.

In embodiments, the flexible tubular bladder is a piece of flexible material that is shaped as a tube. Such tube has a circular cross-section (i.e., is axially symmetric) when expanded but may be collapsed into a partially flattened tube (e.g., ovular or the like) in practice. Such tube has a length, wherein the length determines in part the amount of digester feed that can be added to the apparatus and the residence time of digester feed and slurry in the apparatus. Such tube has a top side and a bottom side, wherein the bottom side is configured to make contact with the ground (wherein “ground” includes any platform upon which the apparatus sits). The top side is not configured to contact the ground. The actual portion of the bottom side of the apparatus that contacts the ground will depend on a variety of factors, such as the amount of digester feed/slurry material in the apparatus and the pressure of the gas in the headspace of the apparatus. Generally, as the pressure of the gas increases, the apparatus adopts a more circular cross-section and less of the bottom side contacts the ground. Also generally, as more digester feed is added to the apparatus the weight of the contents increases and the apparatus adopts a more ovular or flattened circle cross section, with more of the bottom side contacting the ground.

In embodiments, the flexible tubular bladder is shaped as a tube and has a digester feed end and a slurry end. The digester feed end is the end of the tube that is configured to receive digester feed input. The slurry end is the end of the tube that is configured to release slurry from the apparatus. The digester feed end comprises a digester feed inlet port and the slurry end comprises a slurry outlet port, both of which are located on the bottom side of the flexible tubular bladder. Each port comprises an opening in the flexible tubular bladder and may comprise a port sealing mechanism that is configured to connect to an inlet or outlet tube. Such sealing mechanisms may comprise metal or plastic components such as threaded flanges and the like. As described herein, such sealing mechanisms may create gastight, and/or liquid tight connections with the inlet and outlet tubes, or may create non-gastight and/or non-liquid tight connections with the inlet and outlet tubes.

In embodiments, the digester feed end is configured to be sealed with a removable digester feed end seal. By “removable” is meant that the seal can be closed and opened without causing damage to the seal or to the apparatus. For example, the removable digester feed end seal can comprise a single zip or a double zip. A zip or double zip can be an integrated component, such as a metal or plastic zipper that is sewn or welded into the bladder material. A zip or double zip can also be a separate component, such as one or more plastic pipe(s) with a slit to accept the bladder material. In embodiments, the flexible tubular bladder may comprise a beading that is present at the edges of the material to facilitate reversible sealing. A welded seal is not removable provided that such a seal could only be opened by damaging the seal or the apparatus.

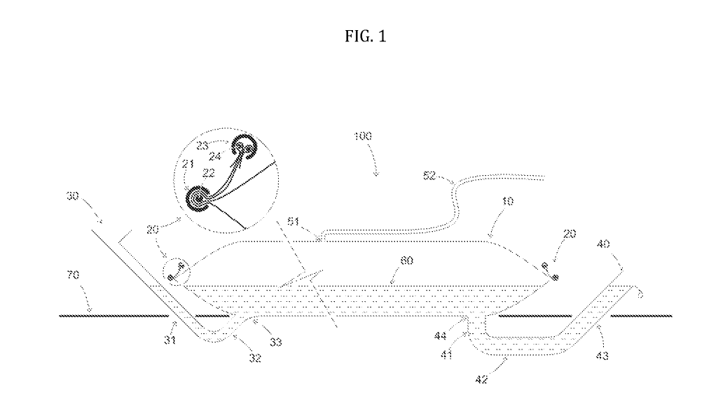

An example of a double zip is installed as follows. A length at the end of the flexible tubular bladder (e.g., 10-30 cm) is flattened and rolled around a small diameter rod. The rod is inserted into a pipe having a slit lengthwise, such that the flexible tubular bladder materials enters and exits the pipe through the slit. A length of the flexible tubular bladder (e.g., 5-20 cm) exits the pipe through the slit and terminates in a beading. The beading is then inserted into a second pipe also having a slit lengthwise. An example of such a double zip is shown in FIG. 1, which is described below.

In embodiments, the slurry end is configured to be sealed with a removable slurry end seal. For any particular apparatus, the removable slurry end seal may be the same type of seal as the removable digester feed end seal (e.g., they are both double zips), or may be a different type from the removable digester feed end seal (e.g., one is a double zip and one is a single integrated zipper). In embodiments, the slurry end is sealed with a single zip, which facilitates easy opening to remove unwanted materials such as dehydrated substrate, and further facilitates operation of the removable seal as a pressure release mechanism.

In embodiments, the removable seal functions as a safety valve that opens (i.e., fails to remain sealed) when the pressure within the apparatus reaches a critical level. Such a safety valve prevents damage to the apparatus and particularly to the flexible tubular bladder when biogas pressure reaches a critical level. Furthermore, such a safety valve prevents injury to people and animals in the surrounding area.

In embodiments, the removable seal provides easy access to the interior of the flexible tubular bladder. This facilitates easy pre-loading of digester feed (e.g., during installation or after purging), and also facilitates easy removal of scum or other materials (e.g., if the apparatus becomes dehydrated).

In embodiments, the digester feed end is sealed with a non-removable seal, provided that the slurry end is sealed with a removable seal. In embodiments, the slurry end is sealed with a non-removable seal, provided that the digester feed end is sealed with a removable seal. Examples of non-removable seals include welded seals, stitched seals, and the like. In embodiments, one (but not both) of the digester feed end or the slurry end is a continuous material and has no opening (sealed or unsealed, removable or non-removable).

In embodiments, the flexible tubular bladder is made from a single piece of flexible material wherein two edges are sewn, welded, or otherwise sealed such that the material forms the tubular shape described herein. The flexible tubular bladder may be made from more than one piece of material, in which case all of the pieces are sewn, welded, or otherwise sealed into the desired shape.

A variety of materials are appropriate for preparing the flexible tubular bladder. In embodiments, the flexible tubular bladder is made of a flexible film. By “film” is meant to include woven fabrics, monolithic thin films, composite materials, and the like. The thickness of the material may be less than 10, 5, 4, 3, 2, 1, 0.7, 0.5, 0.3, or 0.1 mm. In embodiments the thickness of the material may be greater than 0.05, 0.1, 0.3, 0.5, 0.7, 1, 2, 3, 4, or 5 mm. The material may comprise a plastic (i.e., polymer) film, such as PET, HDPE, LDPE, or the like. The material may comprise a woven material prepared from natural cotton, canvas, a synthetic material such as rayon, polyester, or the like, or combinations thereof. The material may comprise a composite such as metal impregnated fabrics or carbon fiber impregnated fabrics. In embodiments, the material is transparent, or partial transparent (e.g., translucent), or opaque, or a combination thereof (e.g., opaque over most of the area but having transparent windows to allow viewing of the contents). In embodiments, the material is UV-opaque or partially UV-opaque. In embodiments, the material comprises multiple layers such as a fabric layer and a plastic layer, or the like.

In embodiments, the flexible film is a non-elastic material. By “non-elastic” is meant that the material undergoes linear stretching of less than or equal to 50, 40, 30, 20, 10, 5, or 1% under load prior to failure (e.g., bursting, tearing, etc.), wherein the linear stretching is measured by the length of the material prior to stretch compared with the length of the material upon failure. Cotton, canvas, or similar woven fabrics are examples that may be non-elastic.

In some embodiments, the flexible film is an elastic material. Examples of such materials include rubber, plastic materials such as PET, and rubberized materials. Generally, elastic materials undergo linear stretching of greater than or equal to 1, 5, 10, 20, 30, or 50% under load prior to failure.

In embodiments, the apparatus comprise a digester feed inlet tube that connects to the digester feed inlet port. Such connection, as explained herein, may be gastight, liquid tight, both, or neither gastight nor liquid tight. The connection is configured, however, to become gas- and liquid-tight upon use of the apparatus. The digester feed inlet tube extends beyond the digester feed end of the flexible tubular bladder and is configured to receive digester feed (which digester feed may be in the form of a slurry, a liquid, or a solid, as described herein). In embodiments, the digester feed inlet tube comprises a horizontal section, a feed section that connects to the horizontal section at an oblique angle, and a rise section that connects to the horizontal section substantially at a right angle, wherein the rise section forms the connection of the digester feed inlet tube to the digester feed inlet port. The length and the angles of intersections of the various sections can vary according to the terrain, the size of the apparatus, etc. In embodiments, the feed section comprises the section of the digester feed inlet tube that first receives digester feed from the user, and may terminate in a funnel to minimize spillage when digester feed is fed to the apparatus. The feed section may also comprise a cover over the terminal orifice to prevent release of odor or to prevent undesired access to the apparatus (e.g., by children, etc.). The feed section descends and intersects the horizontal section, forming an oblique angle with the horizontal section. Such oblique angle is, for example, greater than or equal to 90, 100, 110, 120, 130, 140, or 150 degrees. In embodiments the oblique angle is less than or equal to 160, 150, 140, 130, 120, or 110 degrees. The feed section may be greater than or equal to about 10, 20, 30, 40, 50, 60, 70, 80, 90, or 100 cm in length, provided that it terminates at a point higher than the slurry outlet tube (see below). The horizontal section is substantially horizontal and therefore parallel to the plane of the apparatus (i.e., parallel to the ground upon which the apparatus sits). The horizontal section may be greater than or equal to about 10, 20, 30, 40, 50, 60, 70, 80, 90, or 100 cm in length. The horizontal section intersects the rise section at an angle that is substantially a right angle, wherein “substantially a right angle” includes embodiments that are slightly larger than 90 degrees. For example, the intersection may be within the range 90-110 degrees or 90-120 degrees. The rise section ascends and forms the connection with the digester feed inlet port. The length of the rise section may be greater than or equal to about 1, 2, 5, 8, 10, 15, or 20 cm. In embodiments, the rise section is less than or equal to about 20, 15, 10, 8, 5, or 2 cm. Because of the rise section, the horizontal section is disposed below the level of the bottom side of the flexible tubular bladder. Thus, where the bottom side of the flexible tubular bladder rests on the ground, portions of the digester feed inlet tube (e.g., the horizontal section and rise section and part of the feed section) are disposed in a channel cut into the ground. The channel is generally cut to match the dimensions of the digester feed inlet tube.

In embodiments, the diameter of the digester feed inlet tube is greater than or equal to about 5, 10, 15, or 20 cm, or is less than or equal to about 25, 20, 15, or 10 cm.

The digester feed inlet tube may be a single tube that is bent or otherwise forced into the horizontal, feed, and rise sections, or may be a combination of two or more tubes with connectors (e.g., couplers) between the tubes. The digester feed inlet tube may be made of any appropriate material, such as plastic (e.g., PVC, PET, etc.), metal, ceramic, or combinations thereof.

In embodiments, the digester feed inlet tube consists of two sections—a descending section and an ascending section. The descending section functions similarly to the feed section described above, and all of the above disclosure relating to the feed section applies equally to the descending section. The ascending section intersects the descending section to form an angle in the range 45-135 degrees, or 50-130 degrees, or 60-120 degrees, such as an angle greater than or equal to 60, 70, 80, 90, 100, or 110 degrees, or less than or equal to 120, 110, 100, 90, 80, or 70 degrees. The ascending section ascends and forms the connection with the digester feed inlet port. The length of the ascending section may be greater than or equal to about 1, 2, 5, 8, 10, 15, or 20 cm.

In embodiments, either or both of the digester feed inlet tube and the slurry outlet tube is a single section from the opening to the inlet or outlet port. By single section is meant that there are no sharp angles or turns in the tube, or, where there is a turn, such turn is continuous. For example, a single section tube can be a J-shaped (or U-shaped) tube that has an opening for digester feed input or slurry output, and an end to connect to the digester feed inlet port or the slurry outlet port. Other suitable tube shapes can be used as appropriate.

In embodiments, the apparatus comprise a slurry outlet tube that connects to the slurry outlet port. Such connection, as explained herein, may be gastight, liquid tight, both, or neither gastight nor liquid tight. The connection is configured, however, to become gas- and liquid-tight upon use of the apparatus. The slurry outlet tube extends beyond the slurry end of the flexible tubular bladder and is configured to discharge slurry. In embodiments, the slurry outlet tube comprises a horizontal section, a takeoff section that connects to the horizontal section at an oblique angle, and a drop section that connects to the horizontal section substantially at a right angle, wherein the drop section forms the connection of the slurry outlet tube to the slurry outlet port. The length and the angles of intersections of the various sections can vary according to the terrain, the size of the apparatus, etc. In embodiments, the takeoff section comprises the section of the slurry outlet tube that discharges slurry. The takeoff section may also comprise a cover over the terminal orifice to prevent undesired access to the apparatus. The takeoff section descends and intersects the horizontal section, forming an oblique angle with the horizontal section. Such oblique angle is, for example, greater than or equal to 90, 100, 110, 120, 130, 140, or 150 degrees. In embodiments the oblique angle is less than or equal to 160, 150, 140, 130, 120, or 110 degrees. In embodiments, the oblique angle is the same as that for the digester feed inlet tube, and in embodiments the oblique angle is different as that for the digester feed inlet tube. The takeoff section may be greater than or equal to about 10, 20, 30, 40, 50, 60, 70, 80, 90, or 100 cm in length, provided that it terminates at a point lower than the terminus of the digester feed inlet tube (see above). The horizontal section is substantially horizontal and therefore parallel to the plane of the apparatus (i.e., parallel to the ground upon which the apparatus sits). The horizontal section may be greater than or equal to about 10, 20, 30, 40, 50, 60, 70, 80, 90, or 100 cm in length. The horizontal section intersects the drop section at an angle that is substantially a right angle (where substantially a right angle is as defined above). The drop section descends from (and forms the connection with) the slurry outlet port. The length of the drop section may be greater than or equal to about 1, 2, 5, 8, 10, 15, or 20 cm. In embodiments, the drop section is less than or equal to about 20, 15, 10, 8, 5, or 2 cm. Because of the drop section, the horizontal section is disposed below the level of the bottom side of the flexible tubular bladder. Thus, where the bottom side of the flexible tubular bladder rests on the ground, portions of the slurry outlet tube (e.g., the horizontal section and drop section and part of the takeoff section) are disposed in a channel cut into the ground. The channel is generally cut to match the dimensions of the slurry outlet tube.

In embodiments, the diameter of the slurry outlet tube is greater than or equal to about 10, 15, or 20 cm, or is less than or equal to about 30, 25, 20, or 15 cm.

The height of the terminus of the slurry outlet tube (as measured from the level of the slurry outlet port) limits the depth of the substrate within the apparatus. In embodiments, the apparatus is configured such that the slurry outlet tube terminates at a point that is equal to or less than 30, 25, 20, 15, or 10 cm above the level of the slurry outlet port. In embodiments, the slurry outlet tube allows regulation of the pressure within the apparatus. Increased gas pressure pushes downward on the substrate, and forces substrate to exit via the terminus of the slurry outlet tube. Thus, in embodiments, the slurry outlet tube terminates at a point such that that prevents failure of the reversible seal(s) (i.e., the termination point is low enough such that gas pressure is maintained below the pressure required to cause the reversible seal to fail.

The slurry outlet tube may be a single tube that is bent or otherwise forced into the horizontal, takeoff, and drop sections, or may be a combination of two or more tubes with connectors (e.g., couplers) between the tubes. The slurry outlet tube may be made of any appropriate material, such as plastic (e.g., PVC, PET, etc.), metal, ceramic, or combinations thereof.

In embodiments, the terminus of the slurry outlet tube is at a lower elevation above the ground compared with the terminus of the digester feed inlet tube. This arrangement ensures that the contents of the apparatus do not leak out of the digester feed inlet tube (i.e., the lowest outlet point of the entire apparatus is the slurry outlet tube).

In embodiments, the digester feed inlet tube diameter is smaller than the slurry outlet tube diameter. The slurry outlet tube may be greater than or equal to 10, 15, 20, 25, 30, 40, or 50% greater in diameter compared with the diameter of the digester feed inlet tube. This arrangement ensures that the apparatus does not become blocked by solids at the slurry side. In other embodiments, the digester feed inlet tube diameter is equal to the slurry outlet tube diameter. In other embodiments, the digester feed inlet tube diameter is greater than the slurry outlet tube diameter. The digester feed inlet tube may be greater than or equal to 10, 15, 20, 25, 30, 40, or 50% greater in diameter compared with the diameter of the slurry outlet tube.

Biogas produced within the flexible tubular bladder is trapped at the upper portion (i.e., the headspace above the substrate level) of the flexible tubular bladder. The gas may be stored in the upper portion until needed. In embodiments, the flexible tubular bladder comprises a gas outlet port on the top side. The gas outlet port comprises an opening in the flexible tubular bladder and a port sealing mechanism that is configured to connect to a gas outlet tube. The connection forms a gastight seal, and may comprise metal and/or plastic components to effectuate such a seal. Examples include O-rings and threaded flanges. The gas outlet port provides access to the upper portion of the flexible tubular bladder, and allows accumulated biogas to be removed.

In embodiments, the apparatus further comprises an auxiliary biogas collection container connected to the gas outlet tube. An example is a flexible bag that is gastight and is configured to receive gas from the gas outlet tube. Alternatively, the gas outlet tube may be connected to a burn-off device that burns biogas as it is produced. In embodiments, the apparatus contains a pressure-regulated outlet system that releases biogas from the apparatus (to either an auxiliary collection container or to a burn-off device) when the pressure within the device reaches a critical level (e.g., the pressure is such that the apparatus is in danger of bursting).

Biogas production is most effective at elevated temperatures, provided that the temperature is not so great as to sterilize the contents of the apparatus. In some embodiments, the apparatus comprises a system for regulating (e.g., elevating) the temperature of the substrate within the flexible tubular bladder. For example, the apparatus comprises a greenhouse configured to enclose the flexible tubular bladder. The greenhouse may rest on the ground surrounding the flexible tubular bladder, and have access ports configured to allow the various inlet and outlet tubes to exit the greenhouse. In embodiments, the greenhouse is a tunnel (i.e., an elongate half cylinder) with a digester feed end and slurry end. In embodiments, the digester feed end and slurry ends are partially or completely removable such that the greenhouse can be configured to allow the desired amount of airflow through the space around the flexible tubular bladder. This configuration ensures that the apparatus will not overheat and sterilize the contents of the apparatus. As another example, the apparatus comprises one or more heating pipes within or under the flexible tubular bladder. The heating pipes can be water-filled and can be connected to a heated water source (or a heater for heating water within the pipes), or the heating pipes can be filled with another similar heating liquid or gas (e.g., air). The heating pipes may also be connected to a pump or fan to circulate the heated liquid or gas through the pipes. In an embodiment, the heating pipes are routed below the flexible tubular bladder such that the flexible tubular bladder sits upon the pipes. In another embodiment the heating pipes are routed through the flexible tubular bladder such that they contact the substrate within the bladder.

In embodiments, weights may be positioned on top of the flexible tubular bladder. Such weights may comprise, for example, natural objects such as tree trunks, or man-made objects such as tires. The weights serve, for example, to regulate the outlet pressure of the biogas.

In embodiments, the apparatus is suitable for biogas production from digester feed. Digester feed includes human and animal excrement, as well as yard clippings and other agricultural waste, as well as mixtures thereof. In embodiments the digester feed can be agricultural products that are grown specifically for use in the digester. Such materials may be referred to as a “crop”. Examples of such include algae, water hyacinth, and the like. In embodiments such materials are relatively high in protein content. In embodiments the digester feed is primarily cow manure and agriculture waste. The contents of the digester feed can be varied over time during use of the apparatus.

The overall size of the apparatus will vary depending on expected and determined usage. For single-family use, the width of the flexible tubular bladder may be about 1-2 meters, such as greater than or equal to 100, 110, 120, 130, 140, 150, 160, 170, 180, or 190 cm, or less than or equal to 200, 190, 180, 170, 160, 150, 140, 130, 120, or 110 cm. The length of the flexible tubular bladder may be about 2-6 meters, such as greater than or equal to 2, 2.5, 3, 3.5, 4, 4.5, 5, or 5.5 meters, or less than or equal to about 6, 5.5, 5, 4.5, 4, 3.5, 3, or 2.5 meters. For large demands, such as in schools or on farms, the width of the bladder may be about 1-3 meters, such as greater than or equal to 1, 1.5, 2, or 2.5 meters, or less than or equal to about 3, 2.5, 2, or 1.5 meters. The length of the bladder of the larger device can be 3-8 meters, such as greater than or equal to about 3, 4, 5, 6, or 7 meters, or less than or equal to about 8, 7, 6, 5, or 4 meters. In embodiments, the flexible tubular bladder is rectangular in plan layout, but other shapes are within the scope of the invention.

Referring to FIG. 1, apparatus 100 is shown. Flexible tubular bladder 10 terminates in double zip 20, which comprises first tube 21 comprising a lengthwise slit, rope 22, and a second tube 23 comprising a lengthwise slit. The flexible tubular bladder material terminates in cord 24 that is inserted into second tube 23 when forming the removable seal. Cord 24 runs around the circumference of the flexible tubular bladder and provides a convenient way for interfacing with the removable seal. Flexible tubular bladder 10 also has digester feed inlet tube 30 and slurry outlet tube 40, and further has gas outlet tube 50. Digester feed inlet tube 30 comprises descending section 31 and ascending section 32, and interfaces with digester feed inlet port 33. Slurry outlet tube 40 comprises drop section 41, horizontal section 42, and takeoff section 43, and interfaces with slurry outlet port 44. Gas outlet tube 50 interfaces with gas outlet port 51. When filled and in operation, the apparatus will have a liquid level 60. Ground level 70 is indicated.

Using the apparatus described herein, the invention provides a method for anaerobically digesting digester feed with biogas production. In an embodiment, the method comprises the steps of: (i) conveying the digester feed through a digester feed inlet tube and into a flexible tubular bladder via a digester feed inlet port, wherein the digester feed inlet port is located on a bottom side of the flexible tubular bladder at a digester feed end; (ii) causing the digester feed to travel from the digester feed end of the flexible tubular bladder to a slurry end of the flexible tubular bladder over a period of time, wherein digestion of digester feed and production of biogas occurs during the period of time; and (iii) removing slurry from the flexible tubular bladder through a slurry outlet tube connected to a slurry outlet port, wherein the slurry outlet port is located on the bottom side of the flexible tubular bladder at the slurry end.

In embodiments, the causing of (ii) comprises conveying additional digester feed into the flexible tubular bladder. Thus, the method involves continuous-flow operation of the apparatus. Digester feed that is added to the digester feed end of the apparatus pushes the contents of the flexible tubular bladder toward the slurry end. As the material transits through the bladder, degradation and biogas production occurs. Alternatively, the causing of (ii) may involve positioning the apparatus on a gently sloping ground. Alternatively, the causing of (ii) may involve manually lifting the flexible tubular bladder at certain positions over time (e.g., at the digester feed end first, then at the centre of the bladder, then nearer to the slurry end).

At optimal transit rates, the degradation is complete when the slurry reaches the slurry end and exits through the slurry outlet port. Non-optimal transit rates may be too fast (in which case the slurry is still undergoing biogas production) or may be too slow (in which case the biogas production finishes but the slurry has not yet reached the slurry end. The apparatus can operate at optimal or non-optimal transit rates.

In embodiments, the method comprises maintaining the digester feed and slurry in the tubular bladder at an average temperature of 25 degrees Celcius or greater during the period of time (i.e., while the digester feed and slurry is inside the flexible tubular bladder). Such maintaining may be carried out actively (e.g., by incorporating heating pipes into the apparatus) or passively (e.g., by using a greenhouse as described herein).

In embodiments, the method comprises removing biogas from the flexible tubular bladder via a gas outlet tube connected to a gas outlet port, wherein the gas outlet port is located on a top side of the flexible tubular bladder. The gas may be removed to an auxiliary storage device or burned immediately.

In embodiments, the method comprises first adding cow manure so as to “seed” the contents of the apparatus with appropriate digesting bacteria, and then adding other sources of digester feed such as human excrement or agricultural waste. In embodiments the apparatus may be periodically “re-seeded” by adding a load of cow manure to the apparatus.

The apparatus may also be operated in a batch mode, in which digester feed is added through either the digester feed inlet tube or by opening the re-sealable seal at the digester feed end. After biogas production, the slurry may be removed from the slurry outlet tube or by opening the re-sealable seal at the slurry end.

The apparatus can be disassembled into a conveniently-transported package, particularly since the flexible tubular bladder comprises a flexible film. Thus, in embodiments, there is provided a kit comprising the flexible tubular bladder, the digester feed inlet tube, the slurry outlet tube, and the gas outlet tube. The kit may further comprise a digester feed end seal and a slurry end seal for reversibly sealing the digester feed end and the slurry end. The kit may further comprise a greenhouse or heating pipes. The kit may further comprise an auxiliary gas storage device. The kit may further comprise instructions for installation and/or operation of the apparatus.

The apparatus of the invention represents a low-cost and highly effective alternative to traditional biogas production devices.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described herein. Throughout this disclosure, ranges and multiple numerical values are provided, and this statement serves as antecedent basis to select any one or multiple of such ranges or values as a specific embodiment.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

What is claimed is:

1. An apparatus for anaerobic digestion of digester feed, the apparatus comprising:
(a) a flexible tubular bladder comprising:
(i) a top side comprising a gas outlet port;
(ii) a bottom side comprising a digester feed inlet port and a slurry outlet port;
(iii) a digester feed end sealed with a digester feed end seal; and
(iv) a slurry end sealed with a slurry end seal, provided that at least one of the digester feed end seal and the slurry end seal is a removable seal;
(b) a digester feed inlet tube connected to the digester feed inlet port;
(c) a slurry outlet tube connected to the slurry outlet port; and
(d) a gas outlet tube connected to the gas outlet port.

2. The apparatus of claim 1, wherein the connection of the inlet tube to the inlet port is not gastight, and wherein the connection of the outlet tube to the outlet port is not gastight.

3. The apparatus of claim 1, wherein the digester feed end seal is not gastight and is not liquid tight, and wherein the slurry end seal is not gastight and is not liquid tight.

4. The apparatus of claim 1, wherein the digester feed inlet tube has a diameter, the slurry outlet tube has a diameter, and the digester feed inlet tube diameter is equal to or smaller than the slurry outlet tube diameter.

5. The apparatus of claim 1, wherein the digester feed inlet tube comprises a horizontal section, a feed section that connects to the horizontal section at an oblique angle, and a rise section that connects to the horizontal section at substantially a right angle, wherein the rise section forms the connection of the digester feed inlet tube to the digester feed inlet port.

6. The apparatus of claim 1, wherein the slurry outlet tube comprises a horizontal section, a takeoff section that connects to the horizontal section at an oblique angle, and a drop section that connects to the horizontal section at substantially a right angle, wherein the drop section forms the connection of the slurry outlet tube to the slurry outlet port.

7. The apparatus of claim 1, comprising a greenhouse configured to enclose the tubular container.

8. The apparatus of claim 1, comprising one or more heating pipes within or under the flexible tubular bladder.

9. A method for anaerobically digesting digester feed with biogas production, comprising conveying the digester feed into an apparatus according to claim 1.

10. A method for anaerobically digesting digester feed with biogas production, comprising the steps of:
(i) conveying the digester feed through a digester feed inlet tube and into a flexible tubular bladder via a digester feed inlet port, wherein the digester feed inlet port is located on a bottom side of the flexible tubular bladder at a digester feed end;
(ii) causing the digester feed to travel from the digester feed end of the flexible tubular bladder to a slurry end of the flexible tubular bladder over a period of time, wherein digestion of digester feed and production of biogas occurs during the period of time; and
(iii) removing slurry from the flexible tubular bladder through a slurry outlet tube connected to a slurry outlet port, wherein the slurry outlet port is located on the bottom side of the flexible tubular bladder at the a slurry end.
